SEQUENCE LISTING (42 sequences)

SEQ ID NO: 1 - DNA, 412 bp, Homo sapiens
acagtgggcagcccgattttctgctgagtaggcgctgtgatttcagaatgtctgggcgaggtaaaggtggcaaggggctgggtaagggaggcgccaagcgccaccggaaggtgctgcgggacaatatccaaggcattacaaagccggcgattcgccgtctcgcccgacgtgggggcgtcaagcgcatttctggtctcatctacgaggagacccggggagtcctcaaagtcttcctggagaacgtgatccgtgacgcggtgacttacacggagcacgccaagcgcaagaccgtcacggccatggatgtggtgtacgcgctgaaacgccagggtcgcaccctttatggtttcggcggttgagctgtccccacagcttctctacagactccaaaaggcccttttcagggccccca

SEQ ID NO: 2 - protein, 103 aa, Homo sapiens [sequence omitted]

SEQ ID NO: 3 - DNA, 431 bp, Homo sapiens
atgcctgaaccctctaagtctgctccagcccctaaaaagggttctaagaaggctatcactaaggcgcagaagaaggatggtaagaagcgtaagcgcagccgcaaggagagctattctatctatgtgtacaaggttctgaagcaggtccaccccgacaccggcatctcatccaaggccatggggatcatgaattccttcgtcaacgacatcttcgagcgcatcgcgggcgaggcttctcgcctggctcactacaataagcgctcgaccatcacctccagggagattcagacggctgtgcgcctgctgctgcctggggagctggctaagcatgctgtgtccgagggcactaaggcagttaccaagtacactagctctaaataagtgcttatgtaagcacttccaaacccaaaggctcttttcagagccaccta

SEQ ID NO: 4 - protein, 126 aa, Homo sapiens
MPEPSKSAPAPKKGSKKAITKAQKKDGKKRKRSRKESYSIYVYKVLKQVHPDTGISSKAMGIMNSFVNDIFERIAGEASRLAHYNKRSTITSREIQTAVRLLLPGELAKHAVSEGTKAVTKYTSSK

SEQ ID NO: 5 - DNA, 1937 bp, Homo sapiens
ggccgcagtttctggagggagccgctgcgggtctttccctcactcgtcctccgcgcgtcgccgctcttcggttctgctctgtccgccgccatggcccaagctgacatcgcgctgatcggattggccgtcatgggccagaacttaattctgaacatgaatgaccacggctttgtggtctgtgcttttaataggactgtctccaaagttgatgatttcttggccaatgaggcaaagggaaccaaagtggtgggtgcccagtccctgaaagagatggtctccaagctgaagaagccccggcggatcatcctcctggtgaaggctgggcaagctgtggatgatttcatcgagaaattggtaccattgttggatactggtgacatcatcattgacggaggaaattctgaatatagggacaccacaagacggtgccgagacctcaaggccaagggaattttatttgtggggagcggagtcagtggtggagaggaaggggcccggtatggcccatcgctcatgccaggagggaacaaagaagcgtggccccacatcaagaccatcttccaaggcattgctgcaaaagtgggaactggagaaccctgctgtgactgggtgggagatgagggagcaggccacttcgtgaagatggtgcacaacgggatagagtatggggacatgcagctgatctgtgaggcataccacctgatgaaagacgtgctgggcatggcgcaggacgagatggcccaggcctttgaggattggaataagacagagctagactcattcctgattgaaatcacagccaatattctcaagttccaagacaccgatggcaaacacctgctgccaaagatcagggacagcgcggggcagaagggcacagggaagtggaccgccatctccgccctggaatacggcgtacccgtcaccctcattggagaagctgtctttgctcggtgcttatcatctctgaaggatgagagaattcaagctagcaaaaagctgaagggtccccagaagttccagtttgatggtgataagaaatcattcctggaggacattcggaaggcactctacgcttccaagatcatctcttacgctcaaggctttatgctgctaaggcaggcagccaccgagtttggctggactctcaattatggtggcatcgccctgatgtggagagggggctgcatcattagaagtgtattcctaggaaagataaaggatgcatttgatcgaaacccggaacttcagaacctcctactggacgacttctttaagtcagctgttgaaaactgccaggactcctggcggcgggcagtcagcactggggtccaggctggcattcccatgccctgttttaccactgccctctccttctatgacgggtacagacatgagatgcttccagccagcctcatccaggctcagcgggattacttcggggctcacacctatgaactcttggccaaaccagggcagtttatccacaccaactggacaggccatggtggcaccgtgtcatcctcgtcatacaatgcctgatcatgctgctcctgtcaccctccacgattccacagaccaggacattccatgtgcctcatggcactgccacctggccctttgccctattttctgttcagttttttaaaagtgttgtaagagactcctgaggaagacacacagtttatttgtaaagtagctctgtgagagccaccatgccctctgcccttgcctcttgggactgaccaggagctgctcatgtgcgtgagagtgggaaccatctccttgcggcagtggcttccgcgtgccccgtgtgctggtgcggttcccatcacgcagacaggaagggtgtttgcgcactctgatcaactggaacctctgtatcatgcggctgaattccctttttcctttactcaataaaagctacatcacactga

SEQ ID NO: 6 - protein, 483 aa, Homo sapiens
MAQADIALIGLAVMGQNLILNMNDHGFVVCAFNRTVSKVDDFLANEAKGTKVVGAQSLKEMVSKLKKPRRIILLVKAGQAVDDFIEKLVPLLDTGDIIIDGGNSEYRDTTRRCRDLKAKGILFVGSGVSGGEEGARYGPSLMPGGNKEAWPHIKTIFQGIAAKVGTGEPCCDWVGDEGAGHFVKMVHNGIEYGDMQLICEAYHLMKDVLGMAQDEMAQAFEDWNKTELDSFLIEITANILKFQDTDGKHLLPKIRDSAGQKGTGKWTAISALEYGVPVTLIGEAVFARCLSSLKDERIQASKKLKGPQKFQFDGDKKSFLEDIRKALYASKIISYAQGFMLLRQAATEFGWTLNYGGIALMWRGGCIIRSVFLGKIKDAFDRNPELQNLLLDDFFKSAVENCQDSWRRAVSTGVQAGIPMPCFTTALSFYDGYRHEMLPASLIQAQRDYFGAHTYELLAKPGQFIHTNWTGHGGTVSSSSYNA

SEQ ID NO: 7 - DNA, 1428 bp, Homo sapiens
acagagttgggagtgactccagagcctcctgcaagatgctgttgattctgctgtcagtggccttgctggccctgagctcagctcagaacttaaatgaagatgtcagccaggaagaatctccctccctaatagcaggaaatccacaaggagcacccccacaaggaggcaacaaacctcaaggtcccccatctcctccaggaaagccacaaggaccacccccacaaggaggcaaccagcctcaaggtcccccacctcctccaggaaagccacaaggaccacccccacaaggaggcaacaaacctcaaggtcccccacctccaggaaagccacaaggaccacccccacaaggagacaagtcccgaagtccccgatctcctccaggaaagccacaaggaccacccccacaaggaggcaaccagcctcaaggtcctccacctcctccaggaaagccacaaggaccacccccacaaggaggcaacaaacctcaaggtcccccacctccaggaaagccacaaggaccacccccacaaggagacaacaagtcccgaagttctcgatctcctccaggaaagccacaaggaccacccccacaaggaggcaaccagccccaaggtcccccacctcctccaggaaagccacaaggaccacccccacaaggaggcaacaaacctcaaggtcccccacctccaggaaagccacaaggaccacccccacaaggagacaacaagtcccaaagtgcccgatctcctccaggaaagccacaaggaccacccccacaaggaggcaaccagccccaaggtcccccacctcctccaggaaagccacaaggaccacccccacaaggaggcaacaaacctcaaggtcccccacctccaggaaagccacaaggaccacccccacaaggaggcagcaagtcccgaagttctcgatctcctccaggaaagccacaaggaccacccccacaaggaggcaaccagcctcaaggtcccccacctcctccaggaaagccacaaggaccacccccacaaggaggcaacaaacctcaaggtcccccacctccaggaaagccacaaggaccacccccacaaggaggcagcaagtcccgaagtgcccgatctcctccaggaaagccacaaggaccaccccaacaagaaggcaacaatcctcaaggtcccccacctccagcaggaggcaatccccagcagcctcaggcacctcctgctggacagccccagggaccaccacgccctcctcaagggggcagaccttccagacctccccagtgacagcctccccagtcatctaggattcaatgacaggaagtgaataagaagatgagagtgattcaaatgattcaaatgccatgacattggaaaaaggtcatcatagctctaacttctgtataccaataaaataattagcttgcaa

SEQ ID NO: 8 - protein, 416 aa, Homo sapiens
MLLILLSVALLALSSAQNLNEDVSQEESPSLIAGNPQGAPPQGGNKPQGPPSPPGKPQGPPPQGGNQPQGPPPPPGKPQGPPPQGGNKPQGPPPPGKPQGPPPQGDKSRSPRSPPGKPQGPPPQGGNQPQGPPPPPGKPQGPPPQGGNKPQGPPPPGKPQGPPPQGDNKSRSSRSPPGKPQGPPPQGGNQPQGPPPPPGKPQGPPPQGGNKPQGPPPPGKPQGPPPQGDNKSQSARSPPGKPQGPPPQGGNQPQGPPPPPGKPQGPPPQGGNKPQGPPPPGKPQGPPPQGGSKSRSSRSPPGKPQGPPPQGGNQPQGPPPPPGKPQGPPPQGGNKPQGPPPPGKPQGPPPQGGSKSRSARSPPGKPQGPPQQEGNNPQGPPPPAGGNPQQPQAPPAGQPQGPPRPPQGGRPSRPPQ

SEQ ID NO: 9 - DNA, 437 bp, Homo sapiens
atgccggaggtgtcatctaaaggtgctaccatttccaagaagggctttaagaaagctgtcgttaagacccagaaaaaggaaggcaaaaagcgcaagaggacccgtaaggagagttattctatttacatctacaaagtgctaaagcaggtccatccggacactggcatctcttcgaaagctatgagcattatgaattccttcgtcactgatatctttgagcgtatagcgagcgaggcatcacgtttggctcactacagcaagcgctccaccatttcttccagagagattcagacagcagtgcgcttgctactgccgggagagctggctaaacatgctgtgtctgagggcaccaaggctgtcactaagtacaccagctccaagtaagcctgctaagtaaacgtcatttctaacccaaaggctcttttcagagccactta

SEQ ID NO: 10 - protein, 127 aa, Homo sapiens
MPEVSSKGATISKKGFKKAVVKTQKKEGKKRKRTRKESYSIYIYKVLKQVHPDTGISSKAMSIMNSFVTDIFERIASEASRLAHYSKRSTISSREIQTAVRLLLPGELAKHAVSEGTKAVTKYTSSK

SEQ ID NO: 11 - DNA, 912 bp, Homo sapiens
acagacctgccccgccatgacccggctgacagtcctggccctgctggctggtctgctggcgtcctcgagggccggctccagcccccttttggacatcgttggcggccggaaggcgaggccccgccagttcccgttcctggcctccattcagaatcaaggcaggcacttctgcgggggtgccctgatccatgcccgcttcgtgatgaccgcggccagctgcttccaaagccagaaccccggggttagcaccgtggtgctgggtgcctatgacctgaggcggcgggagaggcagtcccgccagacgttttccatcagcagcatgagcgagaatggctacgacccccagcagaacctgaacgacctgatgctgcttcagctggaccgtgaggccaacctcaccagcagcgtgacgatactgccactgcctctgcagaacgccacggtggaagccggcaccagatgccaggtggccggctgggggagccagcgcagtggggggcgtctctcccgttttcccaggtttgtcaacgtgactgtgacccccgaggaccagtgtcgccccaacaacgtgtgcaccggtgtgctcacccgccgcggtggcatctgcaatggggacgggggcacccccctcgtctgcgagggcctggcccacggcgtggcctccttttccctggggccctgtggccgaggccctgacttcttcacccgagtggcgctcttccgagactggatcgatggtgttctcaacaacccgggaccggggccagcctaggggggcctgtgacctcccatggagcccagccccgccctccacacctccggcgctccgcacccacctcccacggccccgcccctgcccccgctccggccagaggggccctggctgtaataaagaagccgatctctcctctg

SEQ ID NO: 12 - protein, 251 aa, Homo sapiens
MTRLTVLALLAGLLASSRAGSSPLLDIVGGRKARPRQFPFLASIQNQGRHFCGGALIHARFVMTAASCFQSQNPGVSTVVLGAYDLRRRERQSRQTFSISSMSENGYDPQQNLNDLMLLQLDREANLTSSVTILPLPLQNATVEAGTRCQVAGWGSQRSGGRLSRFPRFVNVTVTPEDQCRPNNVCTGVLTRRGGICNGDGGTPLVCEGLAHGVASFSLGPCGRGPDFFTRVALFRDWIDGVLNNPGPGPA

SEQ ID NO: 13 - DNA, 897 bp, Homo sapiens
agagactgcgagaaggaggtcccccacggcccttcaggatgaaagctgcggtgctgaccttggccgtgctcttcctgacggggagccaggctcggcatttctggcagcaagatgaacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccagtgaggcgcccgccgccgccccccttcccggtgctcagaataaacgtttccaaagtggg

SEQ ID NO: 14 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 15 - DNA, 1781 bp, Homo sapiens
cttctcaatatcagcactggattgtagaacttgttgctgattttggcctggcattcaagttaactcttccccttggtatctgtacatacctttgatgtcagtgtttagtacacgtggcttggtcacttcatggctaaaaacgtgcttgtggaagacaagtctggcttggaatataaatagtttctggaaaggacactgacaacttcaaagcaaaatgaagctcttttggttgcttttcaccattgggttctgctgggctcagtattcctcaaatacacaacaaggacgaacatctattgttcatctgtttgaatggcgatgggttgatattgctcttgaatgtgagcgatatttagctcccaagggatttggaggggttcaggtctctccaccaaatgaaaatgttgccattcacaaccctttcagaccttggtgggaaagataccaaccagttagctataaattatgcacaagatctggaaatgaagatgaatttagaaacatggtgactagatgcaacaatgttggggttcgtatttatgtggatgctgtaattaatcatatgtgtggtaatgctgtgagtgcaggaacaagcagtacctgtggaagttacttcaaccctggaagtagggactttccagcagtcccatattctggatgggattttaatgatggtaaatgtaaaactggaagtggagatatcgagaactataatgatgctactcaggtcagagattgtcgtctgtctggtcttctcgatcttgcactggggaaggattatgtgcgttctaagattgccgaatatatgaaccatctcattgacattggtgttgcagggttcagaattgatgcttccaagcacatgtggcctggagacataaaggcaattttggacaaactgcataatctaaacagtaactggttcccggaaggtagtaaacctttcatttaccaggaggtaattgatctgggtggtgagccaattaaaagcagtgactactttggtaatggccgggtgacagaattcaagtatggtgcaaaactcggcacagttattcgcaagtggaatggagagaagatgtcttacttaaagaactggggagaaggttggggtttcatgccttctgacagagcgcttgtctttgtggataaccatgacaatcaacgaggacatggcgctggaggagcctctatacttaccttctgggatgctaggctgtacaaaatggcagttggatttatgcttgctcatccttatggatttacacgagtaatgtcaagctaccgttggccaagatattttgaaaatggaaaagatgttaatgattgggttgggccaccaaatgataatggagtaactaaagaagttactattaatccagacactacttgtggcaatgactgggtctgtgaacatcgatggcgccaaataaggaacatggttaatttccgcaatgtagtggatggccagccttttacaaactggtatgataatgggagcaaccaagtggcttttgggagaggaaacagaggattcattgttttcaacaatgatgactggacattttctttaactttgcaaactggtcttcctgctggcacatactgtgatgtcatttctggagataaaattaatggcaactgcacaggcattaaaatctacgtttctgatgatggcaaagctcatttttctattagtaactctgctgaagatccatttattgcaattcatgctgaatctaaattgtaaaatttaaaattaaatgcaaatccgcaaagca

SEQ ID NO: 16 - protein, 511 aa, Homo sapiens
MKLFWLLFTIGFCWAQYSSNTQQGRTSIVHLFEWRWVDIALECERYLAPKGFGGVQVSPPNENVAIHNPFRPWWERYQPVSYKLCTRSGNEDEFRNMVTRCNNVGVRIYVDAVINHMCGNAVSAGTSSTCGSYFNPGSRDFPAVPYSGWDFNDGKCKTGSGDIENYNDATQVRDCRLSGLLDLALGKDYVRSKIAEYMNHLIDIGVAGFRIDASKHMWPGDIKAILDKLHNLNSNWFPEGSKPFIYQEVIDLGGEPIKSSDYFGNGRVTEFKYGAKLGTVIRKWNGEKMSYLKNWGEGWGFMPSDRALVFVDNHDNQRGHGAGGASILTFWDARLYKMAVGFMLAHPYGFTRVMSSYRWPRYFENGKDVNDWVGPPNDNGVTKEVTINPDTTCGNDWVCEHRWRQIRNMVNFRNVVDGQPFTNWYDNGSNQVAFGRGNRGFIVFNNDDWTFSLTLQTGLPAGTYCDVISGDKINGNCTGIKIYVSDDGKAHFSISNSAEDPFIAIHAESKL

SEQ ID NO: 17 - DNA, 3215 bp, Homo sapiens
gacaatatcaggtgagctgtggaggtggggtccttggaagctggatgacagcagctggcaaggggataagagagcagtgagcccctccctcaaggaggtctggctttatccatagacagggccctctgaggtggggctgaggtacaaagggggattgagcagcccaggagaagagagatgggggttcccttcttctcttctctcagatgcatggtggacttaggaccttgctgggctgggggtctcactgcagagatgaagctgcttctggccctagcagggctcctggccattctggccacgccccagccctctgaaggtgctgctccagctgtcctgggggaggtggacacctcgttggtgctgagctccatggaggaggccaagcagctggtggacaaggcctacaaggagcggcgggaaagcatcaagcagcggcttcgcagcggctcagccagccccatggaactcctatcctacttcaagcagccggtggcagccaccaggacggcggtgagggccgctgactacctgcacgtggctctagacctgctggagaggaagctgcggtccctgtggcgaaggccattcaatgtcactgatgtgctgacgcccgcccagctgaatgtgttgtccaagtcaagcggctgcgcctaccaggacgtgggggtgacttgcccggagcaggacaaataccgcaccatcaccgggatgtgcaacaacagacgcagccccacgctgggggcctccaaccgtgcctttgtgcgctggctgccggcggagtatgaggacggcttctctcttccctacggctggacgcccggggtcaagcgcaacggcttcccggtggctctggctcgcgcggtctccaacgagatcgtgcgcttccccactgatcagctgactccggaccaggagcgctcactcatgttcatgcaatggggccagctgttggaccacgacctcgacttcacccctgagccggccgcccgggcctccttcgtcactggcgtcaactgcgagaccagctgcgttcagcagccgccctgcttcccgctcaagatcccgcccaatgacccccgcatcaagaaccaagccgactgcatcccgttcttccgctcctgcccggcttgccccgggagcaacatcaccatccgcaaccagatcaacgcgctcacttccttcgtggacgccagcatggtgtacggcagcgaggagcccctggccaggaacctgcgcaacatgtccaaccagctggggctgctggccgtcaaccagcgcttccaagacaacggccgggccctgctgccctttgacaacctgcacgatgacccctgtctcctcaccaaccgctcagcgcgcatcccctgcttcctggcaggggacacccgttccagtgagatgcccgagctcacctccatgcacaccctcttacttcgggagcacaaccggctggccacagagctcaagagcctgaaccctaggtgggatggggagaggctctaccaggaagcccggaagatcgtgggggccatggtccagatcatcacttaccgggactacctgcccctggtgctggggccaacggccatgaggaagtacctgcccacgtaccgttcctacaatgactcagtggacccacgcatcgccaacgtcttcaccaatgccttccgctacggccacaccctcatccaacccttcatgttccgcctggacaatcggtaccagcccatggaacccaacccccgtgtccccctcagcagggtcttttttgcctcctggagggtcgtgctggaaggtggcattgaccccatcctccggggcctcatggccacccctgccaagctgaatcgtcagaaccaaattgcagtggatgagatccgggagcgattgtttgagcaggtcatgaggattgggctggacctgcctgctctgaacatgcagcgcagcagggaccacggcctcccaggatacaatgcctggaggcgcttctgtgggctcccgcagcctgaaactgtgggccagctgggcacggtgctgaggaacctgaaattggcgaggaaactgatggagcagtatggcacgcccaacaacatcgacatctggatgggcggcgtgtccgagcctctgaagcgcaaaggccgcgtgggcccactcctcgcctgcatcatcggtacccagttcaggaagctccgggatggtgatcggttttggtgggagaacgagggtgtgttcagcatgcagcagcgacaggccctggcccagatctcattgccccggatcatctgcgacaacacaggcatcaccaccgtgtctaagaacaacatcttcatgtccaactcatatccccgggactttgtcaactgcagtacacttcctgcattgaacctggcttcctggagggaagcctcctagaggccaggtaagggggtgcagcagtgaggggtatatctgggctggccagttggaaccacggagatctccttgccctagatgagcccagccctgttctgggtgcagctgagaaaatgagtgactagacgttcatttgtgtgctcatgtatgtgcgaagtatataaattggcttttcatgcgtgtgtgttgtctgaacatggggagtgtttcatgggttatgtgtatgtgccatttatgtgagtgtgtgtttgtgctgatgagaatactgagtatgtggaaggcagcagagcggactggtgaggagcacagctcaggaactagactgcctgggttccaatcctggctctgtggcttgctagctatgtgaccttgagcaaattaccctccttaaacaagagttttcttccttgtaaattacatctgtcatggtttcttggagggcccacttgtatcctctggttcttcatttattgagcacctactacatgcaaggcactgtactaggcgtgagaagcatatagaggcaagaaagagataccaagatgccatctgtgtcctggttagcagagctggaccagtggtgccttggagggataagccagctgcagctgggctgtgtggttgacttatgggcccagccagccaggctcaggccatggctcccctttttcttcctcaccctgatttcttgcttattcactgaagttctcctgaagaggaactgggcctgttgccctttctgtaccatttatttgctcccaatgtttatgataataaaggcaccgctgatggggacctcc

SEQ ID NO: 18 - protein, 745 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 19 - DNA, 532 bp, Homo sapiens
gagaaaccagagactgtagcaactctggcagggagaagctgtctctgatggcctgaagctgtgggcagctggccaagcctaaccgctataaaaaggagctgcctctcagccctgcatgtctcttgtcagctgtctttcagaagacctggtggggcaagtccgtgggcatcatgttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtagctgagttactgggcccagaggctgggcccctggacatgtacctgcagaataataaagtcatcaatacctcaaaaaaaaaa

SEQ ID NO: 20 - protein, 93 aa, Homo sapiens
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 21 - DNA, 938 bp, Homo sapiens
gttgactaagtcaataatcagaatcagcaggtttgcagtcagattggcagggataagcagcctagctcaggagaagtgagtataaaagccccaggctgggagcagccatcacagaagtccactcattcttggcaggatggcttctcatcgtctgctcctcctctgccttgctggactggtatttgtgtctgaggctggccctacgggcaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggcacttttcattccactttaacttgattttttaaattcccttattgtcccttccaaaaaaaagagaatcaaaattttacaaagaatcaaaggaattctagaaagtatctgggcagaacgctaggagagatccaaatttccattgtcttgcaagcaaagcacgtattaaatatgatctgcagccattaaaaagacacattctgtaaaaaaaaaaaa

SEQ ID NO: 22 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 23 - DNA, 784 bp, Homo sapiens
acagcctctcccagccccagcaagcgacctgtcaggcggccgtggactcagactccggagatgaagcccctgctcctggccgtcagccttggcctcattgctgccctgcaggcccaccacctcctggcctcagacgaggagattcaggatgtgtcagggacgtggtatctgaaggccatgacggtggacagggagttccctgagatgaatctggaatcggtgacacccatgaccctcacgaccctggaagggggcaacctggaagccaaggtcaccatgctgataagtggccggtgccaggaggtgaaggccgtcctggagaaaactgacgagccgggaaaatacacggccgacgggggcaagcacgtggcatacatcatcaggtcgcacgtgaaggaccactacatcttttactgtgagggcgagctgcacgggaagccggtccgaggggtgaagctcgtgggcagagaccccaagaacaacctggaagccttggaggactttgagaaagccgcaggagcccgcggactcagcacggagagcatcctcatccccaggcagagcgaaacctgctctccagggagcgattagggggacaccttggctcctcagcagcccaaggacggcaccatccagcacctccgtcattcacagggacatggaaaaagctccccacccctgcagaacgcggctggctgcaccccttcctaccaccccccgccttccccctgccctgcgccccctctcctggttctccataaagagcttcagcagttcccagtga

SEQ ID NO: 24 - protein, 176 aa, Homo sapiens
MKPLLLAVSLGLIAALQAHHLLASDEEIQDVSGTWYLKAMTVDREFPEMNLESVTPMTLTTLEGGNLEAKVTMLISGRCQEVKAVLEKTDEPGKYTADGGKHVAYIIRSHVKDHYIFYCEGELHGKPVRGVKLVGRDPKNNLEALEDFEKAAGARGLSTESILIPRQSETCSPGSD

SEQ ID NO: 25 - DNA, 586 bp, Homo sapiens
aaacactctgtgtggctcctcggctttgacagagtgcaagacgatgacttgcaaaatgtcgcagctggaacgcaacatagagaccatcatcaacaccttccaccaatactctgtgaagctggggcacccagacaccctgaaccagggggaattcaaagagctggtgcgaaaagatctgcaaaattttctcaagaaggagaataagaatgaaaaggtcatagaacacatcatggaggacctggacacaaatgcagacaagcagctgagcttcgaggagttcatcatgctgatggcgaggctaacctgggcctcccacgagaagatgcacgagggtgacgagggccctggccaccaccataagccaggcctcggggagggcaccccctaagaccacagtggccaagatcacagtggccacggccacggccacagtcatggtggccacggccacagccactaatcaggaggccaggccaccctgcctctacccaaccagggccccggggcctgttatgtcaaactgtcttggctgtggggctaggggctggggccaaataaagtctcttcctccaagtcaaaaaaaaaa

SEQ ID NO: 26 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 27 - DNA, 1054 bp, Homo sapiens
atcccagcagactgtgcagtggggcaaggatttcatgagcatcctcctctaaacgcgtgtcaagacaaaagatgcttcagctttggaaacttgttctcctgtgcggcgtgctcactgggacctcagagtctcttcttgacaatcttggcaatgacctaagcaatgtcgtggataagctggaacctgttcttcacgagggacttgagacagttgacaatactcttaaaggcatccttgagaaactgaaggtcgacctaggagtgcttcagaaatccagtgcttggcaactggccaagcagaaggcccaggaagctgagaaattgctgaacaatgtcatttctaagctgcttccaactaacacggacatttttgggttgaaaatcagcaactccctcatcctggatgtcaaagctgaaccgatcgatgatggcaaaggccttaacctgagcttccctgtcaccgcgaatgtcactgtggccgggcccatcattggccagattatcaacctgaaagcctccttggacctcctgaccgcagtcacaattgaaactgatccccagacacaccagcctgttgccgtcctgggagaatgcgccagtgacccaaccagcatctcactttccttgctggacaaacacagccaaatcatcaacaagttcgtgaatagcgtgatcaacacgctgaaaagcactgtatcctccctgctgcagaaggagatatgtccactgatccgcatcttcatccactccctggatgtgaatgtcattcagcaggtcgtcgataatcctcagcacaaaacccagctgcaaaccctcatctgaagaggacgaatgaggaggaccactgtggtgcatgctgattggttcccagtggcttgccccacccccttatagcatctccctccaggaagctgctgccaccacctaaccagcgtgaaagcctgagtcccaccagaaggaccttcccagataccccttctcctcacagtcagaacagcagcctctacacatgttgtcctgcccctggcaataaaggcccatttctgcacccttaa

SEQ ID NO: 28 - protein, 249 aa, Homo sapiens
MLQLWKLVLLCGVLTGTSESLLDNLGNDLSNVVDKLEPVLHEGLETVDNTLKGILEKLKVDLGVLQKSSAWQLAKQKAQEAEKLLNNVISKLLPTNTDIFGLKISNSLILDVKAEPIDDGKGLNLSFPVTANVTVAGPIIGQIINLKASLDLLTAVTIETDPQTHQPVAVLGECASDPTSISLSLLDKHSQIINKFVNSVINTLKSTVSSLLQKEICPLIRIFIHSLDVNVIQQVVDNPQHKTQLQTLI

SEQ ID NO: 29 - DNA, 622 bp, Homo sapiens
cataaaccctggcgcgctcgcgggccggcactcttctggtccccacagactcagagagaacccaccatggtgctgtctcctgccgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtagccgttcctcctgcccgctgggcctcccaacgggccctcctcccctccttgcaccggcccttcctggtctttgaataaagtctgagtgggcagcaaaaaaaaaaaaaaaaaa

SEQ ID NO: 30 - protein, 142 aa, Homo sapiens
MVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 31 - DNA, 692 bp, Homo sapiens
aaacccctggtacctgagcactgatctgccttggagaacctgatcctgagactccagcaggatgtcttatcaacagcagcagtgcaagcagccctgccagccacctcctgtgtgccccacgccaaagtgcccagagccatgtccacccccgaagtgccctgagccctgcccaccaccaaagtgtccacagccctgcccacctcagcagtgccagcagaaatatcctcctgtgacaccttccccaccctgccagtcaaagtatccaccgaagagcaagtaacagcttcagaattcatcaggaccaagaaaggataaggatatttggctcacctcgttccacagctccaccttcatcttctcatcaaagcctaccatggatacacagggagcttctttctccttagccagtaatctgcccatgatgatccctgacagcaaaaagtttcttttctgaggctgccatactgccactgtccaggtggagactgagcaaaggaagtcctgggctgtgccagctcccagagcttcggaagaaagagcagcagctctctccctgggaaccatcagagaattctgttgatgtgttctgtgtctgtctgtcacctggtcacgagcttctaccacctttgcaattgtcacttatctttcactccctgaataaagtatctatgcatataaaaaaaaaaaaaaaa

SEQ ID NO: 32 - protein, 72 aa, Homo sapiens
MSYQQQQCKQPCQPPPVCPTPKCPEPCPPPKCPEPCPPPKCPQPCPPQQCQQKYPPVTPSPPCQSKYPPKSK

SEQ ID NO: 33 - DNA, 774 bp, Homo sapiens
agggcaagttaagggaatagtggaatgaaggttcatttttcattctcacaaactaatgaaaccctgcttatcttaaaccaacctgctcactggagcagggaggacaggaccagcataaaaggcagggcagagtcgactgttgcttacactttcttctgacataacagtgttcactagcaacctcaaacagacaccatggtgcatctgactcctgaggagaagactgctgtcaatgccctgtggggcaaagtgaacgtggatgcagttggtggtgaggccctgggcagattactggtggtctacccttggacccagaggttctttgagtcctttggggatctgtcctctcctgatgctgttatgggcaaccctaaggtgaaggctcatggcaagaaggtgctaggtgcctttagtgatggcctggctcacctggacaacctcaagggcactttttctcagctgagtgagctgcactgtgacaagctgcacgtggatcctgagaacttcaggctcttgggcaatgtgctggtgtgtgtgctggcccgcaactttggcaaggaattcaccccacaaatgcaggctgcctatcagaaggtggtggctggtgtggctaatgccctggctcacaagtaccattgagatcctggactgtttcctgataaccataagaagaccctatttccctagattctattttctgaacttgggaacacaatgcctacttcaagggtatggcttctgcctaataaagaatgttcagctcaacttcctgat

SEQ ID NO: 34 - protein, 147 aa, Homo sapiens
MVHLTPEEKTAVNALWGKVNVDAVGGEALGRLLVVYPWTQRFFESFGDLSSPDAVMGNPKVKAHGKKVLGAFSDGLAHLDNLKGTFSQLSELHCDKLHVDPENFRLLGNVLVCVLARNFGKEFTPQMQAAYQKVVAGVANALAHKYH

SEQ ID NO: 35 - DNA, 2179 bp, Homo sapiens
gatccgagccccgcctcctccccctgccccgcctctcccatccccgccccgccccgcccggcgacttaacgcgcccccgccccgcgcccggcctcggcagccgcctgtcgccgcgggagcagccgctatctctgtgtgtccgcgtgtgcgcccggtccccgcctgccgcaccatggagagctaccacaagcctgaccagcagaagctgcaggccttgaaggacacggccaaccgcctacgtatcagctccatccaggccaccactgcggcgggctctggccaccccacgtcatgctgcagcgccgcagagatcatggctgtcctctttttccacaccatgcgctacaagtcccaggacccccggaatccgcacaatgaccgctttgtgctctccaagggccatgcagctcccatcctctacgcggtctgggctgaagctggtttcctggccgaggcggagctgctgaacctgaggaagatcagctccgacttggacgggcacccggtcccgaaacaagctttcaccgacgtggccactggctccctgggccagggcctcggggccgcttgtgggatggcctacaccggcaaatacttcgacaaggccagctaccgagtctattgcttgctgggagacggggagctgtcagagggctctgtatgggaggccatggccttcgccagcatctataagctggacaaccttgtggccattctagacatcaatcgcctgggccagagtgacccggccccactgcagcaccagatggacatctaccagaagcggtgcgaggccttcggttggcatgccatcatcgtggatggacacagcgtggaggagctgtgcaaggcctttggccaggccaagcaccagccaacagccatcattgccaagaccttcaagggccgagggatcacgggggtagaagataaggagtcttggcatgggaagcccctccccaaaaacatggctgagcagatcatccaggagatctacagccagatccagagcaaaaagaagatcctggcaacccctccacaggaggacgcaccctcagtggacattgccaacatccgcatgcccagcctgcccagctacaaagttggggacaagatagccacccgcaaggcctacgggcaggcactggccaagctgggccatgccagtgaccgcatcatcgccctggatggggacaccaaaaattccaccttctcggagatcttcaaaaaggagcacccggaccgcttcatcgagtgctacattgctgagcagaacatggtgagcatcgcggtgggctgtgccacccgcaacaggacggtgcccttctgcagcacttttgcagccttcttcacgcgggcctttgaccagattcgcatggccgccatctccgagagcaacatcaacctctgcggctcccactgcggcgtttccatcggggaagacgggccctcccagatggccctagaagatctggctatgtttcggtcagtccccacatcaactgtcttttacccaagtgatggcgttgctacagagaaggcagtggaactagccgccaatacaaagggtatctgcttcatccggaccagccgcccagaaaatgccatcatctataacaacaatgaggacttccaggtcggacaagccaaggtggtcctgaagagcaaggatgaccaggtgaccgttatcggggctggggtgaccctgcacgaggccttggccgctgccgaactgctgaagaaagaaaagatcaacatccgcgtgctggaccccttcaccatcaagcccctggacagaaaactcattctcgacagcgctcgtgccaccaagggcaggatcctcaccgtggaggaccattattatgaaggtggcattggtgaggctgtgtccagtgcagtagtgggcgagcctggcatcactgtcacccacctggcagttaaccgggtaccaagaagtgggaagccggctgagctgctgaagatgtttggtatcgacagggatgccattgcacaagctgtgaggggcctcatcaccaaggcctagggcgggtatgaagtgtggggcgggggtctatacattcctgagattctgggaaaggtgctcaaagatgtactgagaggaggggtaaatatatgttttgagaaaaatgaattggccctgaaaaaaaaaaaaaaaaaa

SEQ ID NO: 36 - protein, 623 aa, Homo sapiens
MESYHKPDQQKLQALKDTANRLRISSIQATTAAGSGHPTSCCSAAEIMAVLFFHTMRYKSQDPRNPHNDRFVLSKGHAAPILYAVWAEAGFLAEAELLNLRKISSDLDGHPVPKQAFTDVATGSLGQGLGAACGMAYTGKYFDKASYRVYCLLGDGELSEGSVWEAMAFASIYKLDNLVAILDINRLGQSDPAPLQHQMDIYQKRCEAFGWHAIIVDGHSVEELCKAFGQAKHQPTAIIAKTFKGRGITGVEDKESWHGKPLPKNMAEQIIQEIYSQIQSKKKILATPPQEDAPSVDIANIRMPSLPSYKVGDKIATRKAYGQALAKLGHASDRIIALDGDTKNSTFSEIFKKEHPDRFIECYIAEQNMVSIAVGCATRNRTVPFCSTFAAFFTRAFDQIRMAAISESNINLCGSHCGVSIGEDGPSQMALEDLAMFRSVPTSTVFYPSDGVATEKAVELAANTKGICFIRTSRPENAIIYNNNEDFQVGQAKVVLKSKDDQVTVIGAGVTLHEALAAAELLKKEKINIRVLDPFTIKPLDRKLILDSARATKGRILTVEDHYYEGGIGEAVSSAVVGEPGITVTHLAVNRVPRSGKPAELLKMFGIDRDAIAQAVRGLITKA

SEQ ID NO: 37 - DNA, 2162 bp, Homo sapiens
cactccctgggctaaacagcatcaccatgtctgttcgatacagctcaagcaagcactactcttcctcccgcagtggaggaggaggaggaggaggaggatgtggaggaggaggaggagtgtcatccctaagaatttctagcagcaaaggctcccttggtggaggatttagctcaggggggttcagtggtggctcttttagccgtgggagctctggtgggggctgctttgggggctcatcaggtggctatggaggattaggaggttttggtggaggtagctttcgtggaagctatggaagtagcagctttggtgggagttatggaggcagctttggagggggcagtttcggaggtggcagctttggtgggggcagctttggtggaggcggctttggtggaggcggctttggaggaggctttggtggtggatttggaggagatggtggccttctctctggaaatgaaaaagtaaccatgcagaatctgaatgaccgcctggcttcctacttggacaaagttcgggctctggaagaatcaaactatgagctggaaggcaaaatcaaggagtggtatgaaaagcatggcaactcacatcagggggagcctcgtgactacagcaaatactacaaaaccatcgatgaccttaaaaatcagattctcaacctaacaactgataatgccaacatcctgcttcagatcgacaatgccaggctggcagctgatgacttcaggctgaagtatgagaatgaggtagctctgcgccagagcgtggaggctgacatcaacggcctgcgtagggtgctggatgagctgaccctgaccaaggctgacctggagatgcaaattgagagcctgactgaagagctggcctatctgaagaagaaccacgaggaggaaatgaaagaccttcgaaatgtgtccactggtgatgtgaatgtggaaatgaatgctgccccgggtgttgatctgactcaacttctgaataacatgagaagccaatatgaacaacttgctgaacaaaaccgcaaagatgctgaagcctggttcaatgaaaagagcaaggaactgactacagaaattgataataacattgaacagatatccagctataaatctgagattactgaattgagacgtaatgtacaagctctggagatagaactacagtcccaactggccttgaaacaatccctggaagcctccttggcagaaacagaaggtcgctactgtgtgcagctctcacagattcaggcccagatatccgctctggaagaacagttgcaacagattcgagctgaaaccgagtgccagaatactgaataccaacaactcctggatattaagatccgactggagaatgaaattcaaacctaccgcagcctgctagaaggagagggaagttccggaggcggcggacgcggcggcggaagtttcggcggcggctacggcggcggaagctccggcggcggaagctccggcggcggccacggcggcggccacggcggcagttccggcggcggctacggaggcggaagctccggcggcggaagctccggcggcggctacgggggcggaagctccagcggcggccacggcggcagttccagcggcggctacggtggtggcagttccggcggcggcggcggcggctacgggggcggcagctccggcggcggcagcagctccggcggcggatacggcggcggcagctccagcggaggccacaagtcctcctcttccgggtccgtgggcgagtcttcatctaagggaccaagatactaacaaaaccagagtaatcaagacaattattgaagaggtggcgcccgacggtagagttctttcatctatggttgaatcagaaaccaagaaacactactattaaactgcatcaagaggaaagagtctcccttcacacagaccattatttacagatgcatggaaaacaaagtctccaagaaaacacttctgtcttgatggtctatggaaatagaccttgaaaataaggtgtctacaaggtgttttgtggtttctgtatttcttcttttcactttaccagaaagtgttctttaatggaaagaaaaacaactttctgttctcatttactaatgaatttcaataaactttcttactgatgcaaactaaaaaaaaaaaaaaaaaaaaaaa

SEQ ID NO: 38 - protein, 584 aa, Homo sapiens
MSVRYSSSKHYSSSRSGGGGGGGGCGGGGGVSSLRISSSKGSLGGGFSSGGFSGGSFSRGSSGGGCFGGSSGGYGGLGGFGGGSFRGSYGSSSFGGSYGGSFGGGSFGGGSFGGGSFGGGGFGGGGFGGGFGGGFGGDGGLLSGNEKVTMQNLNDRLASYLDKVRALEESNYELEGKIKEWYEKHGNSHQGEPRDYSKYYKTIDDLKNQILNLTTDNANILLQIDNARLAADDFRLKYENEVALRQSVEADINGLRRVLDELTLTKADLEMQIESLTEELAYLKKNHEEEMKDLRNVSTGDVNVEMNAAPGVDLTQLLNNMRSQYEQLAEQNRKDAEAWFNEKSKELTTEIDNNIEQISSYKSEITELRRNVQALEIELQSQLALKQSLEASLAETEGRYCVQLSQIQAQISALEEQLQQIRAETECQNTEYQQLLDIKIRLENEIQTYRSLLEGEGSSGGGGRGGGSFGGGYGGGSSGGGSSGGGHGGGHGGSSGGGYGGGSSGGGSSGGGYGGGSSSGGHGGSSSGGYGGGSSGGGGGGYGGGSSGGGSSSGGGYGGGSSSGGHKSSSSGSVGESSSKGPRY

SEQ ID NO: 39 - DNA, 1635 bp, Homo sapiens
aactctatatagggagttcaactggtcacccagagctgtcctgtggcctctgcagctcagcatggctagggtactgggagcacccgttgcactggggttgtggagcctatgctggtctctggccattgccacccctcttcctccgactagtgcccatgggaatgttgctgaaggcgagaccaagccagacccagacgtgactgaacgctgctcagatggctggagctttgatgctaccaccctggatgacaatggaaccatgctgttttttaaaggggagtttgtgtggaagagtcacaaatgggaccgggagttaatctcagagagatggaagaatttccccagccctgtggatgctgcattccgtcaaggtcacaacagtgtctttctgatcaagggggacaaagtctgggtataccctcctgaaaagaaggagaaaggatacccaaagttgctccaagatgaatttcctggaatcccatccccactggatgcagctgtggaatgtcaccgtggagaatgtcaagctgaaggcgtcctcttcttccaaggtgaccgcgagtggttctgggacttggctacgggaaccatgaaggagcgttcctggccagctgttgggaactgctcctctgccctgagatggctgggccgctactactgcttccagggtaaccaattcctgcgcttcgaccctgtcaggggagaggtgcctcccaggtacccgcgggatgtccgagactacttcatgccctgccctggcagaggccatggacacaggaatgggactggccatgggaacagtacccaccatggccctgagtatatgcgctgtagcccacatctagtcttgtctgcactgacgtctgacaaccatggtgccacctatgccttcagtgggacccactactggcgtctggacaccagccgggatggctggcatagctggcccattgctcatcagtggccccagggtccttcagcagtggatgctgccttttcctgggaagaaaaactctatctggtccagggcacccaggtatatgtcttcctgacaaagggaggctataccctagtaagcggttatccgaagcggctggagaaggaagtcgggacccctcatgggattatcctggactctgtggatgcggcctttatctgccctgggtcttctcggctccatatcatggcaggacggcggctgtggtggctggacctgaagtcaggagcccaagccacgtggacagagcttccttggccccatgagaaggtagacggagccttgtgtatggaaaagtcccttggccctaactcatgttccgccaatggtcccggcttgtacctcatccatggtcccaatttgtactgctacagtgatgtggagaaactgaatgcagccaaggcccttccgcaaccccagaatgtgaccagtctcctgggctgcactcactgaggggccttctgacatgagtctggcctggccccacctcctagttcctcataataaagacagattgcttcttcgcttctcactgaggggccttctgacatgagtctggcctggccccacctccccagtttctcataataaagacagattgcttcttcacttgaatcaagggacctaaaaaaaaaaaa

SEQ ID NO: 40 - protein, 462 aa, Homo sapiens
MARVLGAPVALGLWSLCWSLAIATPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 41 - DNA, 4678 bp, Homo sapiens
gcacacagagcagcataaagcccagttgctttgggaagtgtttgggaccagatggattgtagggagtagggtacaatacagtctgttctcctccagctccttctttctgcaacatggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggatcccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggaattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaatgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttctattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaggcattcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtaaactcagcaggtgtggctgaggtaggagtaacagtccctgacaccatcaccgagtggaaggcaggggccttctgcctgtctgaagatgctggacttggtatctcttccactgcctctctccgagccttccagcccttctttgtggagctcacaatgccttactctgtgattcgtggagaggccttcacactcaaggccacggtcctaaactaccttcccaaatgcatccgggtcagtgtgcagctggaagcctctcccgccttcctagctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagagatcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagctcgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcgccctcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagcagccacatttaccaggactgggaaggctgcacaggtgactatccagtcttcagggacattttccagcaaattccaagtggacaacaacaaccgcctgttactgcagcaggtctcattgccagagctgcctggggaatacagcatgaaagtgacaggagaaggatgtgtctacctccagacatccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctaaccatgtgagccggacagaagtcagcagcaaccatgtcttgatttaccttgataaggtgtcaaatcagacactgagcttgttcttcacggttctgcaagatgtcccagtaagagatctgaaaccagccatagtgaaagtctatgattactacgagacggatgagtttgcaattgctgagtacaatgctccttgcagcaaagatcttggaaatgcttgaagaccacaaggctgaaaagtgctttgctggagtcctgttctcagagctccacagaagacacgtgtttttgtatctttaaagacttgatgaataaacactttttctggtcaatgtcaaaaaaaaaaaaaaaaaaaaaaaaa

SEQ ID NO: 42 - protein, 1474 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

(19) United States
(12) Patent Application Publication
(10) Pub. No.: US 2014/0080782 A1
(43) Pub. Date: Mar. 20, 2014

(54) METHODS OF DIAGNOSING AND TREATING PANCREATIC CANCER

(71) Applicants: Aaron Palmon, Moshav Beit Nekofa (IL); Omer Deutsch, Beer-Sheva (IL); Doron Aframian, Jerusalem (IL); Oren Lahav, Herzlia (IL)

(72) Inventors: Aaron Palmon, Moshav Beit Nekofa (IL); Omer Deutsch, Beer-Sheva (IL); Doron Aframian, Jerusalem (IL); Oren Lahav, Herzlia (IL)

(73) Assignee: Hadasit Medical Research Services and Development Ltd., Jerusalem (IL)

(21) Appl. No.: 13/993,713

(22) PCT Filed: Dec. 13, 2011
(86) PCT No.: PCT/IL2011/000937

§ 371 Date: Nov. 27, 2013

Related U.S. Application Data
(60) Provisional application No. 61/422,254, filed on Dec. 13, 2010.

(51) Int. Cl. G01N 33/68; A61K 31/7068; A61K 31/513

(52) U.S. Cl. CPC G01N 33/6893; A61K 31/513; A61K 31/7068
USPC 514/49; 436/501; 435/7.4; 530/389.1; 530/391.1; 435/7.92; 514/274

(57) ABSTRACT

A method of diagnosing pancreatic cancer in a subject is provided. The method comprising determining a level and/or activity of at least one saliva secreted marker in a saliva sample of the subject wherein an alteration in said marker with respect to an unaffected saliva sample is indicative of the pancreatic cancer.

### FIELD AND BACKGROUND OF THE INVENTION

The present invention, in some embodiments thereof, relates to methods of diagnosing and treating pancreatic cancer.

Pancreatic cancer is frequently called the silent killer as it often goes undetected until late in the disease. Pancreatic cancer is the fifth leading cause of cancer death in the United States with 34,000 deaths expected this year alone.

The median survival time for pancreatic cancer is nine to 12 months with an overall survival rate of only 3 percent at five years. The high mortality rate is due in part to the fact that at the time of diagnosis, more than 50 percent of patients with pancreatic cancer are metastatic for the disease.

Additionally, among those for whom the pancreatic tumor can be surgically removed, 50 percent die of recurrent cancer within two years. This suggests that the biology of pancreatic cancer is relatively refractory to current treatments.

Pancreatic cancer first metastasizes to regional lymph nodes, then to the liver, and, less commonly, to the lungs. It can also directly invade surrounding organs such as the small intestines, stomach and large intestines or metastasize to any surface within the abdomen.

Survival from pancreatic cancer has not been improved substantially during the past 30 years, mainly due to difficulties in early diagnostic. Nowadays, early detection of pancreatic cancer for patients at high-risk is done by invasive means (Endoscopic ultrasound combined with fine-needle-aspiration). These methods cause discomfort, require expert team and are very expensive. Therefore, they are not efficient as screening tool.

Additional background art includes PCT Application Nos. WO 2004/090550, WO 2004/055519; U.S. Patent Application Nos. 2010/0136572 and 2010/0279419; Cui et al., International Journal of Cancer (2009) 124(7): 1614-1621; Cui et al., Cancer Investigation (2009) 27(7): 747-755; Djidja et al., Analytical and Bioanalytical Chemistry (2010) 397(2): 587-601; Geetha et al., Journal of Clinical Biochemistry (2006) 39:18-26; Giusti et al., Journal of Proteome Research (2008) 7(9): 4079-4088; Grønborg et al., Molecular & Cellular Proteomics (2006) 5(1): 157-171; Lin et al., Journal of Proteome Research (2006) 5(9): 2169-2176; Polanski et al., Biomarker Insights (2006) 1:1-48; Ralhan et al., Molecular & Cellular Proteomics (2008) 7(6): 1162-1173; Streckfus et al., Journal of Oncology (2009) (ID 737619): 1-11, 20; and Yu et al., Journal of Proteome Research (2005) 4(5): 1742-1751.

### SUMMARY OF THE INVENTION

According to an aspect of some embodiments of the present invention there is provided a method of diagnosing pancreatic cancer in a subject, the method comprising determining a level and/or activity of at least one marker in a saliva sample of the subject, the at least one marker being selected from the group consisting of myeloperoxidase precursor, protein S100-A8, transthyretin precursor, lipocalin-1 precursor, transketolase and keratin type I cytoskeletal 10, wherein an alteration in the marker with respect to an unaffected saliva sample is indicative of the pancreatic cancer.

According to an aspect of some embodiments of the present invention there is provided a method of diagnosing pancreatic cancer in a subject, the method comprising determining a level and/or activity in a biological sample of the subject of at least one marker selected from the group consisting of apolipoprotein A-I precursor, Histone H4, basic salivary proline-rich protein precursor, histone H2B type 1-A, short palate lung and nasal epithelium carcinoma-associated protein 2 precursor and alpha-2-macroglobulin precursor, wherein an alteration in the marker with respect to an unaffected biological sample is indicative of the pancreatic cancer.

According to an aspect of some embodiments of the present invention there is provided a method of diagnosing cancer in a subject, the method comprising determining a level and/or activity in a biological sample of the subject of at least one marker selected from the group consisting of small proline-rich protein 2A and azurocidin precursor, wherein an alteration in the marker with respect to an unaffected biological sample is indicative of the cancer.

According to an aspect of some embodiments of the present invention there is provided a method of diagnosing pancreatic cancer in a subject, the method comprising determining a level and/or activity in a biological sample of the subject of at least one marker selected from the group consisting of histone H2B type 1-B, 6-phosphogluconate dehydrogenase decarboxylating, alpha-amylase 1 precursor, hemoglobin subunit alpha and hemoglobin subunit delta, wherein a downregulation in the marker with respect to an unaffected biological sample is indicative of the pancreatic cancer.

According to an aspect of some embodiments of the present invention there is provided a method of monitoring treatment efficacy of a pancreatic cancer in a subject in need thereof, the method comprising: (a) treating the subject against the pancreatic cancer; and (b) determining a level and/or activity of at least one marker in a saliva sample of the treated subject, the at least one marker being selected from the group consisting of myeloperoxidase precursor, protein S100-A8, transthyretin precursor, lipocalin-1 precursor, transketolase and keratin type I cytoskeletal 10, wherein an alteration in the level and/or activity of the at least one marker with respect to same in a saliva sample taken prior to the treatment, rendering the level and/or activity more similar to that in an unaffected sample, is indicative of an efficacious treatment.

According to an aspect of some embodiments of the present invention there is provided a method of monitoring treatment efficacy of a pancreatic cancer in a subject in need thereof, the method comprising: (a) treating the subject against the pancreatic cancer; and (b) determining a level and/or activity in a biological sample of the subject of at least one marker selected from the group consisting of Histone H4, basic salivary proline-rich protein precursor, histone H2B type 1-A, apolipoprotein A-I precursor, short palate lung and nasal epithelium carcinoma-associated protein 2 precursor and alpha-2-macroglobulin precursor, wherein an alteration in the level and/or activity of the at least one marker with respect to same in a biological sample taken prior to the treatment, rendering the level and/or activity more similar to that in an unaffected sample, is indicative of an efficacious treatment.

According to an aspect of some embodiments of the present invention there is provided a method of monitoring treatment efficacy of a pancreatic cancer in a subject in need thereof, the method comprising: (a) treating the subject against the pancreatic cancer; and (b) determining a level and/or activity in a biological sample of the subject of at least one marker selected from the group consisting of histone H2B type 1-B, 6-phosphogluconate dehydrogenase decarboxylating, alpha-amylase 1 precursor, wherein an upregulation in the level and/or activity of the at least one marker with respect to same in a biological sample taken prior to the treatment, rendering the level and/or activity more similar to that in an unaffected sample, is indicative of an efficacious treatment.

According to an aspect of some embodiments of the present invention there is provided a method of treating pancreatic cancer, the method comprising: (a) diagnosing the pancreatic cancer in a subject in need thereof according to the method of the claimed invention; and (b) treating the subject against the pancreatic cancer.

According to an aspect of some embodiments of the present invention there is provided a kit for diagnosing pancreatic cancer in a subject, the kit comprising a packaging material which comprises at least one agent which specifically determines a level and/or activity of at least one marker selected from the group consisting of myeloperoxidase precursor, protein S100-A8, transthyretin precursor, lipocalin-1 precursor, transketolase, keratin type I cytoskeletal 10, Histone H4, basic salivary proline-rich protein precursor, histone H2B type 1-A, apolipoprotein A-I precursor, short palate lung, nasal epithelium carcinoma-associated protein 2 precursor, alpha-2-macroglobulin precursor, small proline-rich protein 2A, azurocidin precursor, histone H2B type 1-B, 6-phosphogluconate dehydrogenase decarboxylating, alpha-amylase 1 precursor, hemoglobin subunit alpha and hemoglobin subunit delta, in a saliva sample of the subject.

According to an aspect of some embodiments of the present invention there is provided a kit for diagnosing pancreatic cancer in a subject, the kit comprising a packaging material which comprises at least one agent which specifically determines a level and/or activity of at least one marker in a biological sample of the subject, wherein the marker is selected from the group consisting of Histone H4, basic salivary proline-rich protein precursor, histone H2B type 1-A, apolipoprotein A-I precursor, short palate lung and nasal epithelium carcinoma-associated protein 2 precursor and alpha-2-macroglobulin precursor.

According to an aspect of some embodiments of the present invention there is provided a device for diagnosing pancreatic cancer, the device comprising a support and at least one agent for specifically determining a level and/or activity of at least one marker in a biological sample of the subject attached to the support, wherein the marker is selected from the group consisting of myeloperoxidase precursor, protein S100-A8, transthyretin precursor, lipocalin-1 precursor, transketolase, keratin type I cytoskeletal 10, Histone H4, basic salivary proline-rich protein precursor, histone H2B type 1-A, apolipoprotein A-I precursor, short palate lung, nasal epithelium carcinoma-associated protein 2 precursor, alpha-2-macroglobulin precursor, small proline-rich protein 2A, azurocidin precursor, histone H2B type 1-B, 6-phosphogluconate dehydrogenase decarboxylating, alpha-amylase 1 precursor, hemoglobin subunit alpha and hemoglobin subunit delta.

According to some embodiments of the invention, the cancer comprises pancreatic cancer.

According to some embodiments of the invention, the method further comprises removing amylase from the saliva sample.

According to some embodiments of the invention, the removing the amylase comprises: contacting the saliva sample with starch under conditions enabling binding between the amylase and the starch; and separating between the starch-amylase bound complexes and the free components, thereby removing the bound amylase; and collecting the non-bound components.

According to some embodiments of the invention, the at least one agent is an antibody.

According to some embodiments of the invention, the device being a lateral flow device.

According to some embodiments of the invention, the device being a dipstick or a cartridge.

According to some embodiments of the invention, the alteration in the marker comprises an increased activity or expression.

According to some embodiments of the invention, the alteration in the marker comprises a decreased activity or expression.

According to some embodiments of the invention, the marker is selected from the group consisting of histone H2B type 1-B, 6-phosphogluconate dehydrogenase decarboxylating, alpha-amylase 1 precursor, hemoglobin subunit alpha and hemoglobin subunit delta.

According to some embodiments of the invention, the marker is selected from the group consisting of transketolase, keratin type I cytoskeleton 10, hemopexin precursor, alpha 2 macroglobulin precursor.

According to some embodiments of the invention, the determining the level is at the protein level.

According to some embodiments of the invention, the saliva sample comprises unstimulated saliva.

According to some embodiments of the invention, the biological sample comprises a biological fluid.

According to some embodiments of the invention, the biological sample comprises a serum sample.

According to some embodiments of the invention, the biological sample comprises a saliva sample.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DESCRIPTION OF SPECIFIC EMBODIMENTS OF THE INVENTION

The present invention, in some embodiments thereof, relates to methods of diagnosing and treating pancreatic cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Pancreatic cancer often goes undetected until its advanced stages. By the time symptoms occur, diagnosing pancreatic cancer is usually relatively simple (e.g. using blood tests, CT, MRI, ultrasound, biopsy, etc.) however, a cure is rarely possible at that point. Current methods of early diagnosis of pancreatic cancer for patients at high-risk is done by invasive means (e.g. using endoscopic ultrasound combined with fine-needle-aspiration). Thus, simpler non-invasive methods are warranted for early detection of pancreatic cancer.

Whilst reducing the present invention to practice, the present inventors have uncovered that several biomarkers identified in saliva samples of pancreatic cancer patients may serve as accurate predictors of the disease. These biomarkers were shown to be over-expressed by more than 3 fold in oral fluids of pancreatic cancer subjects compared to healthy subjects (see Table 2, in the Examples section which follows). Thus, the present inventors envision that the current set of biomarkers may serve as markers for early diagnosis, screening, therapeutic follow-up and prognosis of pancreatic cancer as well as other cancers.

Thus, according to one aspect of the present invention, there is provided a method of diagnosing pancreatic cancer in a subject, the method comprising determining a level and/or activity in a biological sample of the subject of at least one marker selected from the group consisting of Histone H4, basic salivary proline-rich protein precursor, histone H2B type 1-A, apolipoprotein A-I precursor, short palate lung and nasal epithelium carcinoma-associated protein 2 precursor and alpha-2-macroglobulin precursor, wherein an alteration in the marker with respect to an unaffected biological sample is indicative of the pancreatic cancer.

According to an aspect of the present invention, there is provided a method of diagnosing pancreatic cancer in a subject, the method comprising determining a level and/or activity of at least one marker in a saliva sample of the subject, the at least one marker being selected from the group consisting of myeloperoxidase precursor, protein S100-A8, transthyretin precursor, lipocalin-1 precursor, transketolase and keratin type I cytoskeletal 10, wherein an alteration in the marker with respect to an unaffected saliva sample is indicative of the pancreatic cancer.

According to an aspect of the present invention, there is provided a method of diagnosing cancer in a subject, the method comprising determining a level and/or activity in a biological sample of the subject of at least one marker selected from the group consisting of small proline-rich protein 2A and azurocidin precursor, wherein an alteration in the marker with respect to an unaffected biological sample is indicative of the cancer.

According to an aspect of the present invention, there is provided a method of diagnosing pancreatic cancer in a subject, the method comprising determining a level and/or activity in a biological sample of the subject of at least one marker selected from the group consisting of histone H2B type 1-B, 6-phosphogluconate dehydrogenase decarboxylating, alpha-amylase 1 precursor, hemoglobin subunit alpha and hemoglobin subunit delta, wherein a downregulation in the marker with respect to an unaffected biological sample is indicative of the pancreatic cancer.

As used herein, the term “diagnosing” refers to determining the presence of cancer, such as pancreatic cancer, classifying a cancer (e.g. pancreatic cancer), determining a severity of cancer (grade or stage), monitoring cancer progression, forecasting an outcome of the cancer and/or prospects of recovery, also known as prognosing. The term “detecting” may also optionally encompass any of the above.

The term “cancer” as used herein, refers to a disease or disorder resulting from the proliferation of oncogenically transformed cells.

Non-limiting examples of cancers which can be diagnosed by the method of this aspect of some embodiments of the invention can be any solid or non-solid cancer and/or cancer metastasis, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute—megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

The present invention also contemplates diagnosis of precancers. Precancers are well characterized and known in the art (refer, for example, to Berman J J. and Henson D E., 2003. Classifying the precancers: a metadata approach. BMC Med Inform Decis Mak. 3:8). Classes of precancers amenable to diagnosis via the method of some embodiments of the invention include acquired small or microscopic precancers, acquired large lesions with nuclear atypia, precursor lesions occurring with inherited hyperplastic syndromes that progress to cancer, and acquired diffuse hyperplasias and diffuse metaplasias. Examples of small or microscopic precancers include HGSIL (High grade squamous intraepithelial lesion of uterine cervix), AIN (anal intraepithelial neoplasia), dysplasia of vocal cord, aberrant crypts (of colon), PIN (prostatic intraepithelial neoplasia). Examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Examples of precursor lesions occurring with inherited hyperplastic syndromes that progress to cancer include atypical mole syndrome, C cell adenomatosis and MEA. Examples of acquired diffuse hyperplasias and diffuse metaplasias include AIDS, atypical lymphoid hyperplasia, Paget's disease of bone, post-transplant lymphoproliferative disease and ulcerative colitis.

According to a specific embodiment, the cancer is pancreatic cancer

The term “pancreatic cancer” as used herein, refers to a malignant disease of the pancreas in which at least some of the cells are oncogenically transformed. The term pancreatic cancer encompasses exocrine pancreatic cancer including, for example, pancreatic adenocarcinoma, adenosquamous carcinoma, squamous cell carcinoma, giant cell carcinoma and acinar cell carcinoma, endocrine pancreatic cancer, neuroendocrine pancreatic cancer, islet cell pancreatic cancer and ampullary cancer. Pancreatic cancer may comprise a disease at any stage and spread (i.e. metastatic cancer) including no spread (stage 0), local growth (stage 1), local spread (stage 2), wider spread (stage 3) and confirmed spread (stage 4). According to a specific embodiment, the pancreatic cancer is at an early stage (e.g., asymptomatic, no spread or local growth i.e. stage 0-1).

As used herein the term “subject” or “subject in need thereof” may refer to male or female subject at any age including a healthy human or animal subject undergoing a routine well-being check up. Alternatively, the subject may be at risk of having cancer e.g. pancreatic cancer (e.g., a genetically predisposed subject, a subject with medical and/or family history of cancer, a subject who has been exposed to carcinogens, occupational hazard, environmental hazard) and/or a subject who exhibits suspicious clinical signs of cancer e.g. pancreatic cancer [e.g., unexplained pain, sweating, unexplained fever, unexplained loss of weight up to anorexia, changes in bowel habits (constipation and/or diarrhea), anemia and/or general weakness].

According to another embodiment, the subject may be a diagnosed cancer (e.g. pancreatic cancer) patient who is performing a routine check-up, in-between treatments (monitoring treatment and disease relapse).

Diagnosis of cancer, e.g. pancreatic cancer, according to the present teachings can be effected by determining a level and/or activity of a at least one marker of the present invention in a biological sample obtained from the subject, wherein the level determined can be correlated with predisposition to, presence or absence of the disease, staging of disease and the like (as detailed above for diagnosis).

As used herein, the term “biological sample” refers to a sample of tissue or fluid isolated from a subject, including but not limited to, whole blood, serum, plasma, cerebrospinal fluids, pancreatic fluids, gastro-intestinal fluids, urine, lymph fluids, various external secretions of the skin, respiratory, intestinal and genitourinary tracts, tears, saliva sputum, milk, blood cells, tumors, organs (e.g. tissue biopsy), neuronal tissue and also samples of in vivo cell culture constituents.

It should be noted that a “biological sample obtained from the subject” may also optionally comprise a sample that has not been physically removed from the subject, as described in greater detail below.

According to an embodiment, the biological sample may contain cells or cell content.

The cells used by the present invention can be any cells which are derived from the subject. Examples include, but are not limited to, blood cells, bone marrow cells, hepatic cells, spleen cells, pancreatic cells, kidney cells, cardiac cells, skin cells (e.g., epithelial cells, fibroblasts, keratinocytes), lymph node cells, and fetal cells such as amniotic cells, placental cells (e.g., fetal trophoblasts) and/or cord blood cells.

According to a specific embodiment the biological fluid is saliva or serum.

As used herein, the term “saliva” refers to the oral fluid typically made up of a combination of secretions from a number of sources (e.g., parotid, submandibular, sublingual, accessory glands, gingival mucosa and buccal mucosa).

The saliva analyzed according to the method of the present invention may be stimulated (e.g. by chewing on a piece of paraffin film or tart candy) or unstimulated. According to one embodiment of this aspect of the present invention, the saliva is unstimulated.

Saliva specimens for testing can be collected following various methods known in the art. Proper conditions for generating unstimulated saliva have been described (Nazaresh and Christiansen, J. Dent. Res. 61: 1158-1162 (1982)). Methods and devices for collecting saliva have also been described. (See also, U.S. Pat. No. 5,910,122 to D'Angelo; U.S. Pat. No. 5,714,341 to Thieme et al.; U.S. Pat. Nos. 5,335,673 and 5,103,836 to Goldstein et al.; U.S. Pat. No. 5,268,148 to Seymour; and U.S. Pat. No. 4,768,238 to Kleinberg et al., incorporated herein in their entirety by reference).

Numerous well known fluid collection methods can be utilized to collect the biological sample from the subject, these include, but are not limited to, fine needle biopsy, needle biopsy, core needle biopsy, surgical biopsy (e.g., pancreatic biopsy) and lavage.

The biological sample (e.g. saliva) may be analyzed immediately following collection of the sample. Alternatively, analysis according to the method of the present invention can be performed on a stored sample (e.g. saliva sample). The biological sample (e.g. saliva sample) for testing can be preserved using methods and apparatuses known in the art (see e.g., U.S. Pat. No. 5,968,746 to Schneider, hereby incorporated in its entirety by reference).

According to a specific embodiments the sample is treated prior to analysis (for example, to reduce viscosity and to remove cellular material e.g. from saliva). Techniques used to remove debris include centrifugation and filtration. For example, the viscosity of saliva can also be reduced by mixing a saliva sample with a cationic quaternary ammonium reagent (see, U.S. Pat. No. 5,112,758 to Fellman et al., incorporated herein in its entirety by reference).

Another method of biological fluid (e.g. saliva) processing is described in U.S. 20100108611 and relates to amylase removal from oral fluids, as well as from other body fluids, such as sweat, lacrimal fluid, gastro-intestinal fluid, pancreatic fluids, serum and urine. This is done using a filtering device loaded with starch, which is the amylase substrate. The fluid containing the amylase is passed through the filter containing amylase substrate, a vast amount of the amylase is bound to its substrate and the resulting filtrate has a correspondingly decreased amount of enzyme.

Regardless of the procedure employed, once a biological sample is obtained the level and/or activity of the marker can be determined and a diagnosis can thus be made.

Markers of the present invention may comprise components (e.g. polypeptides) that are secreted into the biological sample e.g. saliva (i.e. do not require cell lysis for detection), alternatively, the markers may be cell associated (e.g. membrane bound). Exemplary markers of the present invention are provided in Table 1, below.

TABLE 1
List of markers

| | | | Accession | MW |
|---|---|---|---|---|
| No. | Protein Identification | Gene symbol | No./Sequence | (Da) |
| 1 | Histone H4 | HIST4H4 | P62805 | 11360 |
| | | | SEQ ID NOs: 1 | |
| | | | and 2 | |
| 2 | Histone H2B type 1-B | HIST1H2BB | P33778 | 13942 |
| | | | SEQ ID NOs: 3 | |
| | | | and 4 | |
| 3 | 6-phosphogluconate dehydrogenase, | PGD | P52209 | 53106 |
| | decarboxylating | | SEQ ID NOs: 5 | |
| | | | and 6 | |
| 4 | Basic salivary proline-rich protein 2 | PRB2 | P02812 | 40775 |
| | precursor | | SEQ ID NOs: 7 | |
| | | | and 8 | |
| 5 | Histone H2B type 1-A | HIST1H2BA | Q96A08 | 14159 |
| | | | SEQ ID NOs: 9 | |
| | | | and 10 | |
| 6 | Azurocidin precursor | AZU1 | P20160 | 26869 |
| | | | SEQ ID NOs: | |
| | | | 11 and 12 | |
| 7 | Apolipoprotein A-I precursor | APOA1 | P02647 | 30759 |
| | | | SEQ ID NOs: | |
| | | | 13 and 14 | |
| 8 | Alpha-amylase 1 precursor | AMY1 | P04745 | 57731 |
| | | | SEQ ID NOs: | |
| | | | 15 and 16 | |
| 9 | Myeloperoxidase precursor | MPO | P05164 | 83815 |
| | | | SEQ ID NOs: | |
| | | | 17 and 18 | |
| 10 | Protein S100-A8 | S100A8 | P05109 | 10828 |
| | | | SEQ ID NOs: | |
| | | | 19 and 20 | |
| 11 | Transthyretin precursor | TTR | P02766 | 15877 |
| | | | SEQ ID NOs: | |
| | | | 21 and 22 | |
| 12 | Lipocalin-1 precursor | LCN1 | P31025 | 19238 |
| | | | SEQ ID NOs: | |
| | | | 23 and 24 | |
| 13 | Protein S100-A9 | S100A9 | P06702 | 13234 |
| | | | SEQ ID NOs: | |
| | | | 25 and 26 | |
| 14 | Short palate, lung and nasal | BPIFA2 | Q96DR5 | 26995 |
| | epithelium carcinoma-associated | | SEQ ID NOs: | |
| | protein 2 precursor | | 27 and 28 | |
| 15 | Hemoglobin subunit alpha | HBA2 | P69905 | 15248 |
| | | | SEQ ID NOs: | |
| | | | 29 and 30 | |
| 16 | Small proline-rich protein 2A | SPRR2A | P35326 | 7960 |
| | | | SEQ ID NOs: | |
| | | | 31 and 32 | |
| 17 | Hemoglobin subunit delta | HBD | P02042 | 16045 |
| | | | SEQ ID NOs: | |
| | | | 33 and 34 | |
| 18 | Transketolase | TKT | P29401 | 67835 |
| | | | SEQ ID NOs: | |
| | | | 35 and 36 | |
| 19 | Keratin type I cytoskeletal 10 | KRT10 | P13645 | 59475 |
| | | | SEQ ID NOs: | |
| | | | 37 and 38 | |
| 20 | Hemopexin precursor | HPX | P02790 | 51643 |
| | | | SEQ ID NOs: | |
| | | | 39 and 40 | |
| 21 | Alpha-2-macroglobulin precursor | A2M | P01023 | 163174 |
| | | | SEQ ID NOs: | |
| | | | 41 and 42 | |

As used herein, the term “level” refers to expression levels of RNA and/or protein or to DNA copy number of a marker of the present invention.

As used herein, the term “activity” refers to the intrinsic biological activity of the marker of the present invention (e.g. protein activity).

According to one embodiment, diagnosis of a cancer (e.g. pancreatic cancer) can be effected by determining an expression level of a polynucleotide (e.g. RNA or DNA) in a biological sample. Any detection method known in the art may be used in accordance with the present teachings, including:

Northern Blot Analysis:

This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR Analysis:

This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA In Situ Hybridization Stain:

In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the slide is subjected to either a photographic emulsion which reveals signals generated using radio-labeled probes or to a colorimetric reaction which reveals signals generated using enzyme-linked labeled probes.

In Situ RT-PCR Stain:

This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountain View, Calif.).

DNA Microarrays/DNA Chips:

The expression of thousands of genes may be analyzed simultaneously using DNA microarrays, allowing analysis of the complete transcriptional program of an organism during specific developmental processes or physiological responses. DNA microarrays consist of thousands of individual gene sequences attached to closely packed areas on the surface of a support such as a glass microscope slide. Various methods have been developed for preparing DNA microarrays. In one method, an approximately 1 kilobase segment of the coding region of each gene for analysis is individually PCR amplified. A robotic apparatus is employed to apply each amplified DNA sample to closely spaced zones on the surface of a glass microscope slide, which is subsequently processed by thermal and chemical treatment to bind the DNA sequences to the surface of the support and denature them. Typically, such arrays are about 2×2 cm and contain about individual nucleic acids 6000 spots. In a variant of the technique, multiple DNA oligonucleotides, usually 20 nucleotides in length, are synthesized from an initial nucleotide that is covalently bound to the surface of a support, such that tens of thousands of identical oligonucleotides are synthesized in a small square zone on the surface of the support. Multiple oligonucleotide sequences from a single gene are synthesized in neighboring regions of the slide for analysis of expression of that gene. Hence, thousands of genes can be represented on one glass slide. Such arrays of synthetic oligonucleotides may be referred to in the art as “DNA chips”, as opposed to “DNA microarrays”, as described above [Lodish et al. (eds.). Chapter 7.8: DNA Microarrays: Analyzing Genome-Wide Expression. In: Molecular Cell Biology, 4th ed., W. H. Freeman, New York. (2000)].

Oligonucleotide Microarray:

In this method oligonucleotide probes capable of specifically hybridizing with the polynucleotides of the present invention are attached to a solid surface (e.g., a glass wafer). Each oligonucleotide probe is of approximately 20-25 nucleic acids in length. To detect the expression pattern of the polynucleotides of the present invention in a specific cell sample (e.g., blood cells), RNA is extracted from the cell sample using methods known in the art (using e.g., a TRIZOL solution, Gibco BRL, USA). Hybridization can take place using either labeled oligonucleotide probes (e.g., 5′-biotinylated probes) or labeled fragments of complementary DNA (cDNA) or RNA (cRNA). Briefly, double stranded cDNA is prepared from the RNA using reverse transcriptase (RT) (e.g., Superscript II RT), DNA ligase and DNA polymerase I, all according to manufacturer's instructions (Invitrogen Life Technologies, Frederick, Md., USA). To prepare labeled cRNA, the double stranded cDNA is subjected to an in vitro transcription reaction in the presence of biotinylated nucleotides using e.g., the BioArray High Yield RNA Transcript Labeling Kit (Enzo, Diagnostics, Affymetix Santa Clara Calif.). For efficient hybridization the labeled cRNA can be fragmented by incubating the RNA in 40 mM Tris Acetate (pH 8.1), 100 mM potassium acetate and 30 mM magnesium acetate for 35 minutes at 94° C. Following hybridization, the microarray is washed and the hybridization signal is scanned using a confocal laser fluorescence scanner which measures fluorescence intensity emitted by the labeled cRNA bound to the probe arrays.

For example, in the Affymetrix microarray (Affymetrix®, Santa Clara, Calif.) each gene on the array is represented by a series of different oligonucleotide probes, of which, each probe pair consists of a perfect match oligonucleotide and a mismatch oligonucleotide. While the perfect match probe has a sequence exactly complimentary to the particular gene, thus enabling the measurement of the level of expression of the particular gene, the mismatch probe differs from the perfect match probe by a single base substitution at the center base position. The hybridization signal is scanned using the Agilent scanner, and the Microarray Suite software subtracts the non-specific signal resulting from the mismatch probe from the signal resulting from the perfect match probe.

According to another embodiment, diagnosis of a cancer (e.g. pancreatic cancer) can be effected by determining a level and/or activity of a polypeptide in a biological sample (e.g. saliva). This is especially advantageous when the marker is a secreted marker or a cell associated marker (e.g., secreted proteins such as Protein S100-A9 P06702, Alpha-2-macroglobulin precursor P01023, Alpha-amylase P04745, Hemopexin P02790, Lipocalin-1 P31025 or Transthyretin P02766). Expression and/or activity level of particular proteins secreted in the saliva can be determined using methods known in the arts, including:

Enzyme Linked Immunosorbent Assay (ELISA):

This method involves fixation of a sample (e.g., saliva) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western Blot:

This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-Immunoassay (RIA):

In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence Activated Cell Sorting (FACS):

This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical Analysis:

This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In Situ Activity Assay:

According to this method, a chromogenic substrate is applied on the cells containing an active enzyme and the enzyme catalyzes a reaction in which the substrate is decomposed to produce a chromogenic product visible by a light or a fluorescent microscope.

In Vitro Activity Assays:

In these methods the activity of a particular enzyme is measured in a protein mixture extracted from the cells. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

It will be appreciated that a combination of the markers of the present invention may be analyzed in order to diagnose the subject. Accordingly, the present invention anticipates analysis of two markers, three markers, four markers, five markers, six markers or more.

As mentioned, the method of the present invention comprises measuring a level and/or activity of a marker in a biological sample (e.g. saliva) and comparing the measurement with an unaffected biological sample (e.g. saliva) wherein an alteration in the amount of the marker is indicative of the cancer.

As used herein, the phrase “unaffected biological sample” refers to a biological sample taken from a healthy subject or from the same subject prior to the onset of the disease (e.g. pancreatic cancer). Since the characteristics and quantities of the biological sample (e.g. saliva) components depend on, amongst other things, species and age, it is preferable that the non-cancerous control sample is obtained from a subject of the same species, age and from the same sub-population (e.g. smoker/nonsmoker). Alternatively, control data may be taken from databases and literature. It will be appreciated that the control sample may also be taken from the diseased subject at a particular time-point, in order to analyze the progression of the disease.

The term “alteration” as used herein refers to an upregulation (i.e. increased activity and/or expression of the marker) or a downregulation (i.e. decreased activity and/or expression of the marker).

According to the present teachings, an upregulation in the level and/or activity of the markers of the present invention may comprise an increase of at least about 1.5, 2, 3, 4, 5, 6, 7, 8, 9, 10 or more fold in the level and/or activity of the marker in the biological sample (e.g. saliva) as compared to an unaffected biological sample.

According to the present teachings, a downregulation in the level and/or activity of the markers of the present invention may comprise a decrease of at least about 1.5, 2, 3, 4, 5, 6, 7, 8, 9, 10 or more fold in the level and/or activity of the marker in the biological sample (e.g. saliva) as compared to an unaffected biological sample.

According to an embodiment of the present invention, an alteration in the marker comprises an increased activity and/or expression thereof. Exemplary markers which expression and/or activity may be increased, include for example, transketolase, keratin type I cytoskeleton 10, hemopexin precursor and alpha 2 macroglobulin precursor.

According to an embodiment of the present invention, an alteration in the marker comprises a decreased activity and/or expression thereof. Exemplary markers which expression and/or activity may be decreased, include for example, histone H2B type 1-B, 6-phosphogluconate dehydrogenase decarboxylating, alpha-amylase 1 precursor, hemoglobin subunit alpha and hemoglobin subunit delta.

Following analysis of the level and/or activity of the marker, the results are typically recorded and the subject is informed. The diagnosis may be substantiated with other (gold-standard) means. Thus, for example, for pancreatic cancer, these results may be assessed along with other diagnosis and detection methods known in the art including, but not limited to, patient's history (e.g. family history of pancreatic cancer, recent onset of atypical diabetes mellitus, a history of recent but unexplained thrombophlebitis (Trousseau sign), or a previous attack of pancreatitis); physical examination (e.g. Courvoisier sign, which defines the presence of jaundice and a painlessly distended gallbladder, are strongly indicative of pancreatic cancer and may be used to distinguish pancreatic cancer from gallstones); description of symptoms including e.g. tiredness, irritability and difficulty eating because of pain e.g. abdominal pain (pain is present in 80-85% of patients with locally advanced or advanced metastatic disease. The pain is usually felt in the upper abdomen as a dull ache that radiates straight through to the back. Pain may be intermittent and made worse by eating), weight loss (weight loss can be profound and can be associated with anorexia, early satiety, diarrhea or steatorrhea) and/or jaundice (jaundice is often accompanied by pruritus and dark urine. Painful jaundice is present in approximately one-half of patients with locally unresectable disease, while painless jaundice is present in approximately one-half of patients with a potentially resectable and curable lesion); laboratory tests including e.g. blood tests for liver function [can illustrate, for example, a combination of results indicative of bile duct obstruction (raised conjugated bilirubin, γ-glutamyl transpeptidase and alkaline phosphatase levels)] and/or blood tests for specific markers such as CA19-9 (carbohydrate antigen 19.9); imaging studies including computed tomography (CT scan), magnetic resonance imaging (MRI), positron emission tomography (PET scan), ultrasound and/or endoscopic ultrasound (EUS) can be used to identify the location and form of the pancreatic cancer; and biopsy can be used to determine the type and stage of the disease.

Subsequent to diagnosing pancreatic cancer according the present methods, the present invention further provides methods of treating the subject against the pancreatic cancer.

Methods of treating pancreatic cancer are well known in the art and include, without being limited to, surgery (e.g. pancreaticoduodenectomy, distal pancreatectomy or total pancreatectomy), chemotherapy [e.g. 5-fluorouracil (5-FU), Gemcitabine], radiation therapy or a combination of same. Determination of the treatment protocol may be carried out by any physician with skill in the art in view of the diagnosis and disease progression.

According to one aspect of the present invention, there is provided a method of monitoring treatment efficacy of a pancreatic cancer in a subject in need thereof, the method comprising: (a) treating the subject against the pancreatic cancer; and (b) determining a level and/or activity of at least one marker in a saliva sample of the treated subject, the at least one marker being selected from the group consisting of myeloperoxidase precursor, protein S100-A8, transthyretin precursor, lipocalin-1 precursor, transketolase and keratin type I cytoskeletal 10, wherein an alteration in the level and/or activity of the at least one marker with respect to same in a saliva sample taken prior to the treatment, rendering the level and/or activity more similar to that in an unaffected sample, is indicative of an efficacious treatment.

According to one aspect of the present invention, there is provided a method of monitoring treatment efficacy of a pancreatic cancer in a subject in need thereof, the method comprising: (a) treating the subject against the pancreatic cancer; and (b) determining a level and/or activity in a biological sample of the subject of at least one marker selected from the group consisting of Histone H4, basic salivary proline-rich protein precursor, histone H2B type 1-A, apolipoprotein A-I precursor, short palate lung and nasal epithelium carcinoma-associated protein 2 precursor and alpha-2-macroglobulin precursor, wherein an alteration in the level and/or activity of the at least one marker with respect to same in a biological sample taken prior to the treatment, rendering the level and/or activity more similar (e.g., at least 70% similar, 80% similar, 90% similar or more) to that in an unaffected sample, is indicative of an efficacious treatment.

According to one aspect of the present invention, there is provided a method of monitoring treatment efficacy of a pancreatic cancer in a subject in need thereof, the method comprising: (a) treating the subject against the pancreatic cancer; and (b) determining a level and/or activity in a biological sample of the subject of at least one marker selected from the group consisting of histone H2B type 1-B, 6-phosphogluconate dehydrogenase decarboxylating, alpha-amylase 1 precursor, wherein an upregulation in the level and/or activity of the at least one marker with respect to same in a biological sample taken prior to the treatment, rendering the level and/or activity more similar to that in an unaffected sample (e.g., at least 70% similar, 80% similar, 90% similar or more), is indicative of an efficacious treatment.

Thus, according to the present teachings, a treatment is considered effective when the marker level and/or activity is comparable to the level and/or activity of the same marker in an unaffected biological sample. Such an assessment may be carried out by any one of ordinary skill in the art in view of the above teachings.

It will be appreciated that the tools necessary for detecting the markers of the present invention may be provided as a kit, such as an FDA-approved kit, which may contain at least one agent which specifically determines a level and/or activity of at least one marker of the present invention in a biological sample (e.g. saliva). Alternatively, the kit may comprise means for collecting the sample (e.g. saliva) and at least one agent for determining a level and/or activity of the marker packaged separately. In addition, the kit may comprise an imaging reagent packed in another container (e.g., enzymes, buffers, chromogenic substrates, fluorogenic material). The kit may further comprise appropriate buffers and preservatives for improving the shelf-life of the kit.

The kit may be accompanied by instructions for administration. The kit may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration.

According to one embodiment, the agent of the present invention comprises an agent for identifying a marker polypeptide (i.e. marker protein).

The presence and/or level of the marker amino acid sequence can be determined using a marker specific antibody via the formation of an immunocomplex [i.e., a complex formed between the marker antigen (a marker amino acid sequence) present in the biological sample and the marker specific antibody].

The immunocomplex of the present invention can be formed at a variety of temperatures, salt concentration and pH values which may vary depending on the method and the biological sample used and those of skills in the art are capable of adjusting the conditions suitable for the formation of each immunocomplex.

The marker specific antibody used in the immunocomplex of the present invention can be labeled using methods known in the art. It will be appreciated that the labeled antibodies can be primary antibodies (i.e., which bind to the specific antigen, e.g., a marker-specific antigen) with or without secondary antibodies (e.g., labeled goat anti rabbit antibodies, labeled mouse anti human antibody) which bind to the primary antibodies. The antibody can be directly conjugated to a label or can be conjugated to an enzyme.

Antibodies of the present invention can be fluorescently labeled (using a fluorescent dye conjugated to an antibody), radiolabeled (using radiolabeled e.g., ¹²⁵I, antibodies), or conjugated to an enzyme (e.g., horseradish peroxidase or alkaline phosphatase) and used along with a chromogenic substrate to produce a colorimetric reaction. The chromogenic substrates utilized by the enzyme-conjugated antibodies of the present invention include, but are not limited to, AEC, Fast red, ELF-97 substrate [2-(5′-chloro-2-phosphoryloxyphenyl)-6-chloro-4(3H)-quinazolinone], p-nitrophenyl phosphate (PNPP), phenolphthalein diphosphate, and ELF 39-phosphate, BCIP/INT, Vector Red (VR), salmon and magenta phosphate (Avivi C., et al., 1994, J Histochem. Cytochem. 1994; 42: 551-4) for alkaline phosphatase enzyme and Nova Red, diaminobenzidine (DAB), Vector(R) SG substrate, luminol-based chemiluminescent substrate for the peroxidase enzyme. These enzymatic substrates are commercially available from Sigma (St Louis, Mo., USA), Molecular Probes Inc. (Eugene, Oreg., USA), Vector Laboratories Inc. (Burlingame, Calif., USA), Zymed Laboratories Inc. (San Francisco, Calif., USA), Dako Cytomation (Denmark).

Detection of the immunocomplex in a biological sample (e.g. saliva) which may contain soluble markers (e.g., secreted, shedded) or cell bound markers can be performed using fluorescence activated cell sorting (FACS), enzyme linked immunosorbent assay (ELISA), Western blot and radio-immunoassay (RIA) analyses, immunoprecipitation (IP) or by a molecular weight-based approach (as described in further detail hereinabove). Those of skills in the art are capable of determining which to method is suitable for each immunocomplex.

Antibodies of the present invention may also be purchased commercially. Exemplary antibodies which may be used in accordance with the present teachings include, but are not limited to, anti-myeloperoxidase precursor antibody (commercially available from, for example, LifeSpan BioSciences and AbFrontier Co., Ltd.), anti-protein S100-A8 antibody (commercially available from, for example, ABR and Atlas Antibodies), anti-transthyretin precursor antibody (commercially available from, for example, Atlas Antibodies and Sigma-Aldrich), anti-lipocalin-1 precursor antibody (commercially available from, for example, United States Biological), anti-transketolase antibody (commercially available from, for example, AbFrontier Co., Ltd.), anti-keratin type I cytoskeletal 10 antibody (commercially available from, for example, United States Biological), anti-histone H4 antibody (commercially available from, for example, Acris Antibodies GmbH and Cell Sciences), anti-basic salivary proline-rich protein precursor antibody (commercially available from, for example, Biotrend, Rockland and Immunoch), anti-histone H2B type 1-A antibody (commercially available from, for example, Abcam), anti-apolipoprotein A-I precursor antibody (commercially available from, for example, Novus Biologicals and United States Biological), anti-short palate lung antibody (commercially available from, for example, Santa Cruz), anti-nasal epithelium carcinoma-associated protein 2 precursor antibody (commercially available from, for example, Novus and Proteintech Group), anti-alpha-2-macroglobulin precursor antibody (commercially available from, for example, GenWay Biotech, Inc.), anti-small proline-rich protein 2A antibody (commercially available from, for example, LifeSpan Biosc), anti-azurocidin precursor antibody, anti-histone H2B type 1-B antibody (commercially available from, for example, R & D Systems), anti-6-phosphogluconate dehydrogenase decarboxylating antibody (commercially available from, for example, AbFrontier Co., Ltd.), anti-alpha-amylase 1 precursor antibody (commercially available from, for example, United States Biological), anti-hemoglobin subunit alpha antibody (commercially available from, for example, Biorbyt) and anti-hemoglobin subunit delta antibody (commercially available from, for example, MyBioSource).

According to another embodiment of the present invention, the agent of the present invention comprises an agent for identifying a nucleic acid sequence or transcript of the marker.

The presence and/or level of a marker nucleic acid sequence (marker transcript) can be determined using an isolated polynucleotide (e.g., a polynucleotide probe, an oligonucleotide probe/primer) capable of hybridizing to a marker nucleic acid sequence or a portion thereof. Such a polynucleotide can be at any size, such as a short polynucleotide (e.g., of 15-200 bases), and intermediate polynucleotide (e.g., 200-2000 bases) or a long polynucleotide larger of 2000 bases.

The isolated polynucleotide probe used by the present invention can be any directly or indirectly labeled RNA molecule (e.g., RNA oligonucleotide, an in vitro transcribed RNA molecule), DNA molecule (e.g., oligonucleotide, cDNA molecule, genomic molecule) and/or an analogue thereof [e.g., peptide nucleic acid (PNA)] which is specific to the marker RNA transcript of the present invention.

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

The oligonucleotide of the present invention is of at least 17, at least 18, at least 19, at least 20, at least 22, at least 25, at least 30 or at least 40, bases specifically hybridizable with sequence alterations described hereinabove.

The above-described polynucleotides can be employed in a variety of RNA detection methods such as Northern blot analysis, reverse-transcribed PCR (RT-PCR) [e.g., a semi-quantitative RT-PCR, quantitative RT-PCR using e.g., the Light Cycler™ to (Roche)], RNA in situ hybridization (RNA-ISH), in situ RT-PCR stain [e.g., as described in Nuovo G J, et al. 1993, Intracellular localization of polymerase chain reaction (PCR)-amplified hepatitis C cDNA. Am J Surg Pathol. 17: 683-90, and Komminoth P, et al. 1994, Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract., 190: 1017-251 and oligonucleotide microarray analysis [e.g., using the Affymetrix microarray (Affymetrix®, Santa Clara, Calif.)], as described in further detail hereinabove.

According to an embodiment, the kit comprises a single agent (e.g. antibody). According to another embodiment, the kit comprises two, three, four, five or more agents. The agents may be packed in a single package or alternatively, may be provided in separate packagings.

According to one embodiment, the kit may be comprised in a device such as a dipstick or a cartridge (optionally comprised in a housing). For example, for testing a saliva sample, the dipstick or cartilage may be one which the subject places into the mouth and detects a change in a salivary component. The device may comprise any agent capable of specifically detecting the markers of the present invention. For example, the device may comprise one or a combination of monoclonal and polyclonal antibody agents and an indicator for detecting binding. Antibody supports are known in the art. In an embodiment of this invention, antibody supports are absorbent pads to which the antibodies are removably or fixedly attached.

According to one embodiment, the device is a lateral flow device. For example, for testing a saliva sample, the lateral flow device may comprise inlet means for flowing saliva into contact with the agent/s capable of detecting the markers of the present invention. The test device can also include a flow control means for assuring that the test is properly operating. Such flow control means can include control antigens bound to a support which capture detection antibodies as a means of confirming proper flow of sample fluid through the test device. Alternatively, the flow control means can include capture antibodies in the control region which capture the detection antibodies, again indicating that proper flow is taking place within the device.

According to an embodiment, the kit comprises a monoclonal antibody colored conjugate and polyclonal anti-monoclonal antibody coated on a membrane test area. By capillary action, the biological sample (e.g. saliva) migrates over the test area and reacts with the impregnated reagents to form visible colored bands in the test window. The presence of the monoclonal antibody in concentrations above normal will result in the formation of a distinct colored band in the test area thus indicating a positive result for the caner. Conversely, if no line appears in the test area, the test is negative.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find support in the following examples.

### Examples

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Materials and Experimental Procedures

Unstimulated oral fluid (OF) was collected from 15 individuals with pancreatic cancer priority treatment and 15 age and gender matched healthy individuals. Samples were centrifuged, their volume measured and protein concentration was determined. OF samples were pooled based on total protein content into 2 groups and pretreated with amylase removal device, albumin and IgG depletion kit in order to increase visualization and gel resolution and improve quantification abilities in 2-DE and in tag-MS for better identification of disease-specific biomarkers, as previously described [Deutsch, O., et al. Electrophoresis (2008) 29(20): p. 4150-7; Krief, G., et al. Oral Dis. (2011) 17(1): p. 45-52]. Thereafter the samples were analyzed using proteomics approaches [2D-SDS-PAGE, dimethylation followed by LC-MS/MS on the Orbitrap (Thermo) mass spectrometer].

Results

Dimethilation followed by LC-MS/MS revealed overall 182 proteins. Out of these, 21 proteins showed significant changes (above 3 fold, Table 2, below). These differences are in correlation with the 2D-SDS-PAGE which also showed dissimilar pattern of the proteomics maps of the 2 groups.

These findings are in correlation to the significant differences found in OF protein expression of pancreatic cancer patients which may serve as biomarkers for early diagnosis, screening, therapeutic follow-up and prognosis, and also for relapse diagnosis in relation to pancreatic cancer.

TABLE 2
List of differentially expressed proteins (>3x)

| | | Accession | MW | avg Ratio |
|---|---|---|---|---|
| No. | Protein Identification | No. | (Da) | (Patient/Healthy) |
| 1 | Histone H4 | P62805 | 11360 | 0.02 |
| 2 | Histone H2B type 1-B | P33778 | 13942 | 0.03 |
| 3 | 6-phosphogluconate dehydrogenase, | P52209 | 53106 | 0.04 |
| | decarboxylating | | | |
| 4 | Basic salivary proline-rich protein 2 precursor | P02812 | 40775 | 0.05 |
| 5 | Histone H2B type 1-A | Q96A08 | 14159 | 0.06 |
| 6 | Azurocidin precursor | P20160 | 26869 | 0.07 |
| 7 | Apolipoprotein A-I precursor | P02647 | 30759 | 0.08 |
| 8 | Alpha-amylase 1 precursor | P04745 | 57731 | 0.16 |
| 9 | Myeloperoxidase precursor | P05164 | 83815 | 0.16 |
| 10 | Protein S100-A8 | P05109 | 10828 | 0.19 |
| 11 | Transthyretin precursor | P02766 | 15877 | 0.22 |
| 12 | Lipocalin-1 precursor | P31025 | 19238 | 0.23 |
| 13 | Protein S100-A9 | P06702 | 13234 | 0.24 |
| 14 | Short palate, lung and nasal epithelium | Q96DR5 | 26995 | 0.24 |
| | carcinoma-associated protein 2 precursor | | | |
| 15 | Hemoglobin subunit alpha | P69905 | 15248 | 0.25 |
| 16 | Small proline-rich protein 2A | P35326 | 7960 | 0.25 |
| 17 | Hemoglobin subunit delta | P02042 | 16045 | 0.26 |
| 18 | Transketolase | P29401 | 67835 | 3.18 |
| 19 | Keratin type I cytoskeletal 10 | P13645 | 59475 | 4.57 |
| 20 | Hemopexin precursor | P02790 | 51643 | 4.99 |
| 21 | Alpha-2-macroglobulin precursor | P01023 | 163174 | 8.06 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of diagnosing pancreatic cancer in a subject, the method comprising determining a level and/or activity of at least one marker in a saliva sample of the subject, said at least one marker being selected from the group consisting of myeloperoxidase precursor, protein S100-A8, transthyretin precursor, lipocalin-1 precursor, transketolase and keratin type I cytoskeletal 10, wherein an alteration in said marker with respect to an unaffected saliva sample is indicative of the pancreatic cancer.

2-5. (canceled)

6. A method of monitoring treatment efficacy of a pancreatic cancer in a subject in need thereof, the method comprising:
(a) treating the subject against said pancreatic cancer; and
(b) determining a level and/or activity of at least one marker in a saliva sample of the treated subject, said at least one marker being selected from the group consisting of myeloperoxidase precursor, protein S100-A8, transthyretin precursor, lipocalin-1 precursor, transketolase and keratin type I cytoskeletal 10,
wherein an alteration in said level and/or activity of said at least one marker with respect to same in a saliva sample taken prior to said treatment, rendering said level and/or activity more similar to that in an unaffected sample, is indicative of an efficacious treatment.

7. The method of claim 1, further comprising removing amylase from said saliva sample.

8. The method of claim 7, wherein said removing said amylase comprises: contacting the saliva sample with starch under conditions enabling binding between the amylase and the starch; and separating between the starch-amylase bound complexes and the free components, thereby removing the bound amylase; and collecting the non-bound components.

9-10. (canceled)

11. A method of treating pancreatic cancer, the method comprising:
(a) diagnosing the pancreatic cancer in a subject in need thereof according to the method of claim 1; and
(b) treating the subject against the pancreatic cancer.

12. A kit for diagnosing pancreatic cancer in a subject, the kit comprising a packaging material which comprises at least one agent which specifically determines a level and/or activity of at least one marker selected from the group consisting of myeloperoxidase precursor, protein S100-A8, transthyretin precursor, lipocalin-1 precursor, transketolase, keratin type I cytoskeletal 10, Histone H4, basic salivary proline-rich protein precursor, histone H2B type 1-A, apolipoprotein A-I precursor, short palate lung, nasal epithelium carcinoma-associated protein 2 precursor, alpha-2-macroglobulin precursor, small proline-rich protein 2A, azurocidin precursor, histone H2B type 1-B, 6-phosphogluconate dehydrogenase decarboxylating, alpha-amylase 1 precursor, hemoglobin subunit alpha and hemoglobin subunit delta, in a saliva sample of the subject.

13. (canceled)

14. A device for diagnosing pancreatic cancer, the device comprising a support and at least one agent for specifically determining a level and/or activity of at least one marker in a biological sample of the subject attached to said support, wherein said marker is selected from the group consisting of myeloperoxidase precursor, protein S100-A8, transthyretin precursor, lipocalin-1 precursor, transketolase, keratin type I cytoskeletal 10, Histone H4, basic salivary proline-rich protein precursor, histone H2B type 1-A, apolipoprotein A-I precursor, short palate lung, nasal epithelium carcinoma-associated protein 2 precursor, alpha-2-macroglobulin precursor, small proline-rich protein 2A, azurocidin precursor, histone H2B type 1-B, 6-phosphogluconate dehydrogenase decarboxylating, alpha-amylase 1 precursor, hemoglobin subunit alpha and hemoglobin subunit delta.

15. The device of claim 14, wherein said at least one agent is an antibody.

16. The device of claim 14, being a lateral flow device.

17. The device of claim 14, being a dipstick or a cartridge.

18. The method of claim 1, wherein said alteration in said marker comprises an increased activity or expression.

19. The method of claim 1, wherein said alteration in said marker comprises a decreased activity or expression.

20. The method of claim 19, wherein said marker is selected from the group consisting of histone H2B type 1-B, 6-phosphogluconate dehydrogenase decarboxylating, alpha-amylase 1 precursor, hemoglobin subunit alpha and hemoglobin subunit delta.

21. The method of claim 18, wherein said marker is selected from the group consisting of transketolase, keratin type I cytoskeleton 10, hemopexin precursor, alpha 2 macroglobulin precursor.

22. The method of claim 1, wherein said determining said level is at the protein level.

23. The method of claim 1, wherein said saliva sample comprises unstimulated saliva.

24-26. (canceled)
